(19) United States
(12) Patent Application Publication
(10) Pub. No.: US 2014/0141100 A1
(43) Pub. Date: May 22, 2014

(54) Compounds and Methods for Appetite Suppression and Weight Control

(71) Applicants: Benedict T. Marino, Kiawah Island, SC (US); James F. Marino, La Jolla, CA (US)

(72) Inventors: Benedict T. Marino, Kiawah Island, SC (US); James F. Marino, La Jolla, CA (US)

(21) Appl. No.: 14/085,644

(22) Filed: Nov. 20, 2013

Related U.S. Application Data
(60) Provisional application No. 61/728,767, filed on Nov. 20, 2012.

(51) Int. Cl. A61K 38/07; A61K 45/06; A61K 31/167

(52) U.S. Cl. CPC A61K 38/07; A61K 31/167; A61K 45/06
USPC 424/649; 514/4.9; 514/630

(57) ABSTRACT

Disclosed is a method of treating obesity, reducing weight, preventing weight gain, and/or suppressing appetite in a subject in need thereof by administering topically a pharmaceutical compound having a chemotherapeutic agent to at least a surface of the subject's mouth. The method includes inhibiting taste cell reproduction on the surface of the subject's mouth with the compound and reducing sense of taste, appetite for food in the subject, or a combination thereof.

### REFERENCE TO PRIORITY DOCUMENT

This application claims priority of U.S. Provisional Patent Application Ser. No. 61/728,767, entitled “Compounds and Method for Appetite Suppression and Weight Control,” filed Nov. 20, 2012. Priority of the filing date is hereby claimed and the disclosure of the provisional patent application is hereby incorporated by reference.

### BACKGROUND

Obesity is a chronic condition that is characterized by a body mass index (BMI) over 25. Both congenital and environmental factors, such as exercise and eating habits, contribute to the disease. Obesity can lead to a number of complications and diseases, including insulin resistance, Type II diabetes, gallbladder disease, hypertension, cardiovascular disease, hyperlipidemia, sleep apnea, coronary artery disease, knee osteoarthritis, gout, infertility, breast cancer, endometrial cancer, colon cancer and lower back pain.

### SUMMARY

In view of the foregoing, there is a need for new treatments of obesity, controlling caloric intake, and/or appetite suppression. Disclosed herein are compounds and methods for treating obesity, reducing weight, preventing weight gain, providing weight control, and/or suppressing appetite in a subject in need. Provided are methods for the local topical use of drugs that affect cell division within the oral cavity to affect taste cell reproduction for the purpose of appetite suppression and weight control.

In one aspect, disclosed is a method of treating obesity, reducing weight, preventing weight gain, and/or suppressing appetite in a subject in need thereof. The method includes administering topically a pharmaceutical compound having a chemotherapeutic agent to at least a surface of the subject's mouth. The method includes inhibiting taste cell reproduction on the surface of the subject's mouth with the compound; and reducing sense of taste, appetite for food in the subject, or a combination thereof.

The chemotherapeutic agent can include antimetabolites, alkylating agents, coordination compounds, inhibitors of transcription enzymes, topoisomerase inhibitors, DNA minor-groove binding compounds, antimitotic agents, vinca alkyloids, mechlorothamine, cyclophosphamide, ifosfamide, melphalan, chlorambucil, ethyleneimines, methylmelamines, alkyl sulfonates, busulfan, carmustine, folic acid analog, methotrexate, pyrimidine analogs, fluorouracil, cytosine arabinoside, purine analogs, mecaptopurine, thioguanine, azathioprine, vinca alkyloids, vinblastine, vincristine, paclitaxel, colchicine, antitumor antibiotics, actinomycin D, daunorubicin, bleomycin, anti-neoplastic L-asparaginase, cisplatin, carboplatin, adrenocorticosteroids prednisone, dexamethasone, aromatase inhibitors, amino glutethimide, formestane, anastrozole, progestin compounds, hydroxyprogesteron caproate, medroxyprogesterone, anti-estrogen compounds, tamoxifen, topoisomerase inhibitors amsacrine (m-AMSA), mitoxantrone, topotecan, irinotecan, and camptothecin or a combination thereof. The chemotherapeutic agent can be bleomycin or colchicine. The chemotherapeutic agent can be suspended in dimethylsulfoxide. The administering step can be performed at least monthly, bi-weekly, weekly, daily, or bi-daily. The pharmaceutical compound can include a chemotherapeutic agent formulated into a lozenge, dissolvable strip, gum, chewable tablet, hard candy, candy, chewing gum with liquid or gel center, mouthwash, rinse, gel, spray, brushed on solution, paste, gel or powder. Administering can include using an applicator to apply the pharmaceutical compound onto the surface. The surface can include one or more of the tongue, soft palate, upper esophagus and epiglottis. The surface can contain one or more taste buds. The method can further include reducing calorie consumption in response to the reducing the sense of taste. The method can further include discontinuing administration of the pharmaceutical compound allowing for return of the sense of taste. The method can further include administering topically to at least a surface of a subject's mouth a second pharmaceutical compound. The second pharmaceutical compound can include an agent that reverses or impairs the effect of the first pharmaceutical compound.

Other features and advantages should be apparent from the following description of various implementations, which illustrate, by way of example, the principles of the invention.

### DETAILED DESCRIPTION

The sense of taste is mediated by taste receptor cells that are bundled into clusters called taste buds. Taste buds are located around macroscopic structures called papillae that are located on the upper surface of the tongue, soft palate, upper esophagus and epiglottis. When the receptors for taste come into contact with the food dissolved in saliva nerves send signals to the brain that are perceived as salty, sweet, sour, bitter, fatty, metallic, and umami (the Japanese word describing the taste of monosodium glutamate) taste. Taste buds are periodically renewed and in adults, the turnover period is approximately every 10 days. This relatively high turn-over rate of taste buds and their taste cells makes them particularly susceptible to drugs or therapeutic agents that suppress or inhibit cell reproduction. The treatment modality considered herein incorporates topical treatments with various drugs or agents that inhibit, suppress, or otherwise impair one or more phases of the cell cycle, such as mitosis. It should be appreciated that the drug type can vary and includes, for example, antimitotic agents, cancer chemotherapeutic agents or other drugs intended to target cells that are going through phases of the cell cycle.

Described herein are methods for treating obesity, reducing weight, preventing weight gain, controlling appetite and/or otherwise treating a patient in need thereof by administering topically or locally to the oral cavity chemotherapeutic agents to inhibit taste cell growth and reproduction. Inhibiting the perception of taste makes eating food less pleasurable. When food is less pleasurable, patients are less likely to eat and the caloric intake is reduced. When the caloric intake is reduced, patients lose weight. Topical administration of these agents to the oral cavity blunts a patient's sense of taste while minimizing the adverse effects that would otherwise result from the use of these agents systemically. It should be appreciated that depending on the compound(s) used in treatment and the treatment regimen, patients can experience hypogeusia, ageusia and/or dysgeusia. The treatments described herein can result in profound reduction or loss of taste that is reversed upon discontinuation of the treatment regimen.

Various classes of cancer chemotherapeutic agents considered herein include, among others, antimetabolites, agents that react with DNA (e.g., alkylating agents, coordination compounds, etc.), inhibitors of transcription enzymes, topoisomerase inhibitors, DNA minor-groove binding compounds, antimitotic agents (e.g., vinca alkyloids), antitumor antibiotics, hormones, and enzymes. Exemplary alkylating agents include, by way of example and not limitation, mechlorothamine, cyclophosphamide, ifosfamide, melphalan, chlorambucil, ethyleneimines, methylmelamines, alkyl sulfonates (e.g., busulfan), and carmustine. Exemplary antimetabolites include, by way of example and not limitation, folic acid analog methotrexate; pyrimidine analogs fluorouracil, cytosine arabinoside; and purine analogs mecaptopurine, thioguanine, and azathioprine. Exemplary vinca alkyloids include, by way of example and not limitation, vinblastine, vincristine, paclitaxel, and colchicine. Exemplary antitumor antibiotics include, by way of example and not limitation, actinomycin D, daunorubicin, and bleomycin. An exemplary enzyme effective as anti-neoplastic agent is L-asparaginase. Exemplary coordination compounds include, by way of example and not limitation, cisplatin and carboplatin. Exemplary hormones and hormone related compounds include, by way of example and not limitation, adrenocorticosteroids prednisone, and dexamethasone; aromatase inhibitors amino glutethimide, formestane, and anastrozole; progestin compounds hydroxyprogesteron caproate, medroxyprogesterone; and anti-estrogen compound tamoxifen. Exemplary topoisomerase inhibitors include, by way of example and not limitation, amsacrine (m-AMSA); mitoxantrone, topotecan, irinotecan, and camptothecin. These and other useful anti-cancer compounds are described in Merck Index, 13th Ed. (O'Neil, M. J. et al., ed) Merck Publishing Group (2001) and Goodman and Gilmans The Pharmacological Basis of Therapeutics, 10th Edition, Hardman, J. G. and Limbird, L. E. eds., pg. 1381-1287, McGraw Hill, (1996), both of which are incorporated herein by reference.

In some implementations, the local administration of bleomycin can be used to inhibit taste cell replication. Bleomycin is an antibiotic shown to have antitumor activity by selectively inhibiting DNA synthesis. In some implementations, the local administration of traditional anti-mitotic agents including microtubule toxins such as taxol, other taxanes and the vinca alkaloids, paclitaxel, docetaxel, vinblastine, vincristine and vinorelbine can be used to inhibit taste cell replication. In other implementations, the local administration of colchicine or other alkyloid agents can be used to inhibit taste bud replication.

The treatment compound can be applied topically onto the surface of a patient's oral cavity, such as on the tongue, to the location of one or more taste receptors. The treatment compound can inhibit gustatory cells or taste bud receptor cells from reproducing and blunts taste sensation (i.e. hypogeusia). This blunting of taste sensation diminishes the appeal of food to the patient, resulting in decreased caloric intake over time and weight loss. Those of skill in the art are familiar with a variety of modes of administration. Modes of administration considered herein include topical administration in the oral cavity resulting in a local effect on the taste buds and taste cells and minimizing the systemic effects of the active compound. The treatment compound is applied generally in the location where its action is desired or within the localized area of the mouth.

The compounds described herein can be administered to a human patient per se, or in pharmaceutical compositions where they are mixed with other active ingredients, as in combination therapy, or suitable carrier(s) or excipient(s). Techniques for formulation and administration of the compounds of the instant application may be found in “Remington: The Science and Practice of Pharmacy,” 20th ed. (2000). Pharmaceutical compositions may be manufactured in a manner that is itself known, e.g., by means of conventional mixing, dissolving, granulating, dragee-making, levigating, emulsifying, encapsulating, entrapping or compression processes. Pharmaceutical compositions may be formulated in conventional manner using one or more physiologically acceptable carriers comprising excipients and auxiliaries, which facilitate processing of the active compounds into preparations, which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

Delivery of agents can be effected either in the absence or in the presence of a carrier. Topical administration in the presence of a carrier serves various purposes, such as controlled release of biologically active molecules, targeting of biologically active molecules to specific tissues, and facilitating penetration into the mucosal layer. One or more of the chemotherapeutic agents described herein can be used in association with a carrier such as a hydrophobic carrier like dimethylsulfoxide (DMSO). The one or more chemotherapeutic agents described herein can be suspended in a solution or dissolved in a solvent, such as DMSO, to facilitate cell membrane penetration of the agent(s).

One or more of the chemotherapeutic agents described herein can be formulated into a variety of compounds appropriate for topical use in the oral cavity. The formulation can include, but is not limited to, lozenges, dissolvable strips, gum, chewable tablets, hard candies, candies, chewing gums with liquid or gel centers. The compounds described herein can also come in liquid forms such as a mouthwash, rinse, gel, spray that can be administered by a pump, aerosol spray, cup or other similar method. The compounds described herein can also come as a brushed on solution, paste, gel or powder.

In some embodiments, the formulation can include a flavor component to improve the taste of the compound upon initial consumption of the formulation and initiation of treatment. For example, a taste compounds can be incorporated that make the formulation sweeter, saltier, more sour, less bitter, more neutral in taste, or otherwise more initially palatable to patients. In more preferred embodiments, the taste compound is selected from the group comprising glycerine, monosaccharides, disaccharides, oligosaccharides, glycerol monostearate, sorbitol, mannitol, glycerol, xylitol, fructose, high fructose corn syrup, dextrose, lactose, maltose, trehalose, galactose, and artificial sweeteners, including aspartame.

The treatment compound can be topically applied to at least a portion of the tongue or oral cavity monthly, bi-weekly, weekly, daily, bi-daily, tri-daily or other appropriate treatment schedule configured to inhibit taste bud and taste cell reproduction and function. Upon discontinuation of treatment with the agent, the patient's normal sense of taste and food cravings return. The return of taste can occur over a variety of time-periods upon discontinuation of the treatment. In some implementations, a reversing or blocking agent can be applied topically or systemically that enhances the return of taste upon completion of treatment. This agent can be used to limit the effects of or customize the appetite suppression therapy. Such an agent can be useful as well in improving the tolerance of chemotherapy. In some implementations, the effects of antimetabolites can be dampened or “reversed” with folic acid, purine, and pyrimidine. In other implementations, Cox-2 inhibitors can be used to block chemotherapeutic apoptosis.

Administration of a composition to the oral cavity generally results in direct contact of the agents with one or more of the tongue, soft palate, upper esophagus and epiglottis onto which at least a portion of the administered agents pass. Often, the composition can have an effective residence time in the mouth that varies depending on whether a slow-release type method of administration is used or a method that is more fast-acting.

In some implementations administration of the agents is combined with a diet plan or an exercise regime. In other implementations administration of the agents is combined with a diet plan and an exercise regime, e.g., walking, swimming, or other cardiovascular exercise, and/or low impact exercise or other exercise regimens as would be contemplated by those skilled in the art, including weight training, yoga, pilates, aerobics, etc., depending on the needs of individual patients.

Although the application is described in terms of human treatment, it should be appreciated that all mammals are considered herein including humans, non-human primates, cows, dogs, cats, goats, sheep, pigs, rats, mice and rabbits.

An “effective amount” of the treatment as used herein refers to that amount of the compound being administered which will produce to some extent the desired effect, including a reduction in the sense of taste.

The amount of a given agent that will correspond to such an amount will vary depending upon factors such as the particular compound, condition of the patient, the identity (e.g., weight) of the subject or host in need of treatment. Doses employed for adult human treatment can be in the range of 0.002-5000 mg per day. In some implementations, the doses for an adult human being treated can be in the range of 0.1-1500 mg per day. In one implementation, topical 1% bleomycin in dimethylsulfoxide can be applied to the oral mucosa of a patient to inhibit taste cell reproduction on one or more surfaces of the oral mucosa and reduce the sense of taste and/or appetite for food of the patient. In another implementation, topical 1% colchicine ointment gel can be applied to the oral mucosa of a patient to inhibit taste cell reproduction on one or more surfaces of the oral mucosa and reduce the sense of taste and/or appetite for food of the patient. The treatments can be used for the treatment of obesity in the patient, reducing weight of the patient, the prevention of weight gain of the patient and/or suppressing appetite of the patient. In some implementations, the treatment can be applied once daily for 14 consecutive days. It should be appreciated that the treatment can be applied more frequently or less frequently and for a shorter or longer period of time depending upon the weight control/loss results achieved by the treatment. For example, the treatment can be applied once, twice, three times or more per day. The treatment can also be applied monthly, bi-weekly, or weekly. The treatment can be applied over a period of 1, 2, 3, 4, 5, 7, 14, 21 days or 4, 5, 6, 7, 8, 9, 10, 11, 12 or more weeks. The desired dose may conveniently be presented in a single dose or as divided doses administered simultaneously (or over a short period of time) or at appropriate intervals, for example as two, three, four or more sub-doses per day.

While this specification contains many specifics, these should not be construed as limitations on the scope of any embodiment that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

What is claimed is:

1. A method of treating obesity, reducing weight, preventing weight gain, and/or suppressing appetite in a subject in need thereof, comprising:
administering topically a pharmaceutical compound comprising a chemotherapeutic agent to at least a surface of the subject's mouth;
inhibiting taste cell reproduction on the surface of the subject's mouth with the compound; and
reducing sense of taste, appetite for food in the subject, or a combination thereof.

2. The method of claim 1, wherein the chemotherapeutic agent is selected from the group consisting of antimetabolites, alkylating agents, coordination compounds, inhibitors of transcription enzymes, topoisomerase inhibitors, DNA minor-groove binding compounds, antimitotic agents, vinca alkyloids, mechlorothamine, cyclophosphamide, ifosfamide, melphalan, chlorambucil, ethyleneimines, methylmelamines, alkyl sulfonates, busulfan, carmustine, folic acid analog, methotrexate, pyrimidine analogs, fluorouracil, cytosine arabinoside, purine analogs, mecaptopurine, thioguanine, azathioprine, vinca alkyloids, vinblastine, vincristine, paclitaxel, colchicine, antitumor antibiotics, actinomycin D, daunorubicin, bleomycin, anti-neoplastic L-asparaginase, cisplatin, carboplatin, adrenocorticosteroids prednisone, dexamethasone, aromatase inhibitors, amino glutethimide, formestane, anastrozole, progestin compounds, hydroxyprogesteron caproate, medroxyprogesterone, anti-estrogen compounds, tamoxifen, topoisomerase inhibitors amsacrine (m-AMSA), mitoxantrone, topotecan, irinotecan, and camptothecin or a combination thereof.

3. The method of claim 1, wherein the chemotherapeutic agent is bleomycin.

4. The method of claim 1, wherein the chemotherapeutic agent is colchicine.

5. The method of claim 1, wherein the chemotherapeutic agent is suspended in dimethylsulfoxide.

6. The method of claim 1, wherein the administering step is performed at least monthly, bi-weekly, weekly, daily, or bi-daily.

7. The method of claim 1, wherein the pharmaceutical compound comprising a chemotherapeutic agent is formulated into a lozenge, dissolvable strip, gum, chewable tablet, hard candy, candy, chewing gum with liquid or gel center, mouthwash, rinse, gel, spray, brushed on solution, paste, gel or powder.

8. The method of claim 1, wherein administering comprises using an applicator to apply the pharmaceutical compound onto the surface.

9. The method of claim 8, wherein the surface is selected from at least one of the group consisting of the tongue, soft palate, upper esophagus and epiglottis.

10. The method of claim 9, wherein the surface contains one or more taste buds.

11. The method of claim 1, further comprising reducing calorie consumption in response to the reducing the sense of taste.

12. The method of claim 1, further comprising discontinuing administration of the pharmaceutical compound allowing for return of the sense of taste.

13. The method of claim 1, further comprising administering topically to at least a surface of a subject's mouth a second pharmaceutical compound comprising an agent that reverses or impairs the effect of the first pharmaceutical compound.

14. The method of claim 2, wherein the chemotherapeutic agent is suspended in dimethylsulfoxide.

15. The method of claim 3, wherein the chemotherapeutic agent is suspended in dimethylsulfoxide.

16. The method of claim 4, wherein the chemotherapeutic agent is suspended in dimethylsulfoxide.
